(19) United States
(12) Patent Application Publication
(10) Pub. No.: US 2023/0240485 A1
(43) Pub. Date: Aug. 3, 2023

(54) BATHROOM TEMPERATURE SENSOR

(71) Applicant: Toi Labs, Inc., San Franscisco, CA (US)

(72) Inventors: Vikram Kashyap, San Francisco, CA (US); Paul Cristman, San Francisco, CA (US); John Wilson, San Francisco, CA (US)

(73) Assignee: Toi Labs, Inc., San Franscisco, CA (US)

(21) Appl. No.: 18/011,467

(22) PCT Filed: Apr. 30, 2021
(86) PCT No.: PCT/US21/30036

§ 371 Date: Mar. 23, 2023

Related U.S. Application Data
(60) Provisional application No. 63/027,115, filed on May 19, 2020.

(51) Int. Cl. A47K 13/24; E03D 9/08; G01J 5/48; G01J 5/00; G01J 5/04; G01N 33/483

(52) U.S. Cl. CPC A47K 13/24; E03D 9/085; G01J 5/485; G01J 5/0025; G01J 5/041; G01N 33/483

(57) ABSTRACT

Provided is a temperature sensor configured to fit under a seat of a toilet that measures a temperature of a user of the toilet or feces expelled by the user without contacting the user or feces. Also provided is a temperature sensor configured to fit on a bathroom appliance that measures a temperature of a user of the appliance without contacting the user. Additionally provided is a temperature sensor configured to fit on a toilet seat that measures a temperature of skin contacting the seat. Further provided is a method of measuring temperature of a bathroom user, the method comprising using any of the above-described temperature sensors to measure the user's temperature.

### CROSS-REFERENCE TO RELATED APPLICATIONS

This application claims the benefit of U.S. Provisional Application No. 63/027,115, filed May 19, 2020, and incorporated by reference herein in its entirety.

### BACKGROUND OF THE INVENTION

### (1) Field of the Invention

The present application generally relates to temperature measurements of a bathroom user. More specifically, the application provides temperature sensors configured to fit on a bathroom appliance that measures the temperature of the user of the appliance.

### (2) Description of the Related Art

PCT Patent Applications PCT/US2018/26618 and PCT/US2020/019383 provide biomonitoring devices for analyzing various aspects of bathroom use, including analysis of excreta and user presence and identification.

A body temperature measurement is a basic measurement of health status of an individual. Measurements can be made orally, rectally, in the ear, in the armpit, on the skin, or without contacting the body using e.g., an infrared (IR) sensor or thermal imaging (Canadian, 2014). While a normal temperature measurement varies by the location of the temperature sensor/thermometer, all of the above temperature measurement regimes provide accurate readings that can reliably determine whether the individual is in the normal range or has a fever (Allegaert et al, 2014; Yao et al., 2007; Bland and Husney, 2019; Mogensen et al., 2018; Canadian, 2014).

Various non-touch thermometers are currently available, including the ThermoFlash, the Braun No touch+touch forehead thermometer, the OMEGA DX-6635, the Avita TS31, the FORA IR 42, the iHealth Thermometer, the Briutcare, the Medescan, and the Innovo FR201 Severa.

The temperature measurement methods currently available generally require human activity—e.g., inserting a thermometer under the tongue or in the ear, or directing the non-contact thermometer to the forehead.

There is a need for passive thermometry, e.g., to obtain multiple measurements in patients or nursing home residents without utilizing valuable nurse or aid effort. The present invention satisfies that need.

### BRIEF SUMMARY OF THE INVENTION

The present invention is directed to temperature sensors for measuring the temperature of a user of a bathroom.

Thus, in some embodiments, provided is a temperature sensor configured to fit under a seat of a toilet that measures a temperature of a user of the toilet or feces expelled by the user without contacting the user or feces.

Also provided is a temperature sensor configured to fit on a bathroom appliance that measures a temperature of a user of the appliance without contacting the user.

Additionally provided is a temperature sensor configured to fit on a toilet seat that measures a temperature of skin contacting the seat.

Further provided is a method of measuring temperature of a bathroom user, the method comprising using any of the above-described temperature sensors to measure the user's temperature.

### BRIEF DESCRIPTION OF THE SEVERAL VIEWS OF THE DRAWINGS

FIG. 1 is an exploded view of a temperature sensor of the present invention.

FIG. 2 is a perspective view of a temperature sensor of the present invention.

FIG. 3 is a perspective view of a toilet seat comprising a temperature sensor of the present invention and an excreta analysis device.

### DETAILED DESCRIPTION OF THE INVENTION

Provided herewith are devices and methods for passively measuring temperature of a bathroom user.

Thus, in some embodiments, provided is a temperature sensor configured to fit under a seat of a toilet that measures a temperature of a user of the toilet or feces expelled by the user without contacting the user or feces.

Any contactless thermometric method known in the art can be utilized in the temperature sensors provided herein, including but not limited to infrared (IR) sensing or thermal imaging. In some embodiments, the temperature is measured using at least one IR sensor.

In various embodiments, the temperature sensor is capable of recording the temperature in a dataset and transmitting the dataset to a computing device, for example through a printed circuit board (PCB) operatively connected thereto.

As used herein, “operatively connected” means the recited components are connected in a way to perform a designated function. Here, the PCB can be operatively connected to the temperature sensor to record the temperature and transmit the recorded temperature to a computing device.

In some embodiments, the temperature sensor can be attached to a toilet seat or a toilet rim. In some of these embodiments, the temperature sensor is integrated into a toilet seat or toilet rim. In other embodiments, the temperature sensor is attached to, or integrated into, the toilet seat lid/cover. In those latter embodiments, the temperature sensor can measure forehead temperature when the user is facing the toilet.

In various embodiments, the temperature sensor further comprises a mounting bracket for mounting the sensor to the toilet seat or toilet rim. In additional embodiments, the temperature sensor further comprises an IR translucent cover and/or a housing or casing.

In other embodiments, the temperature sensor further comprises an IR translucent disposable cover to protect and keep the sensor clean. In some of these embodiments, the cover can be secured in place over the sensor, e.g., with features on the sensor and/or the cover to align and/or lock the cover to the sensor.

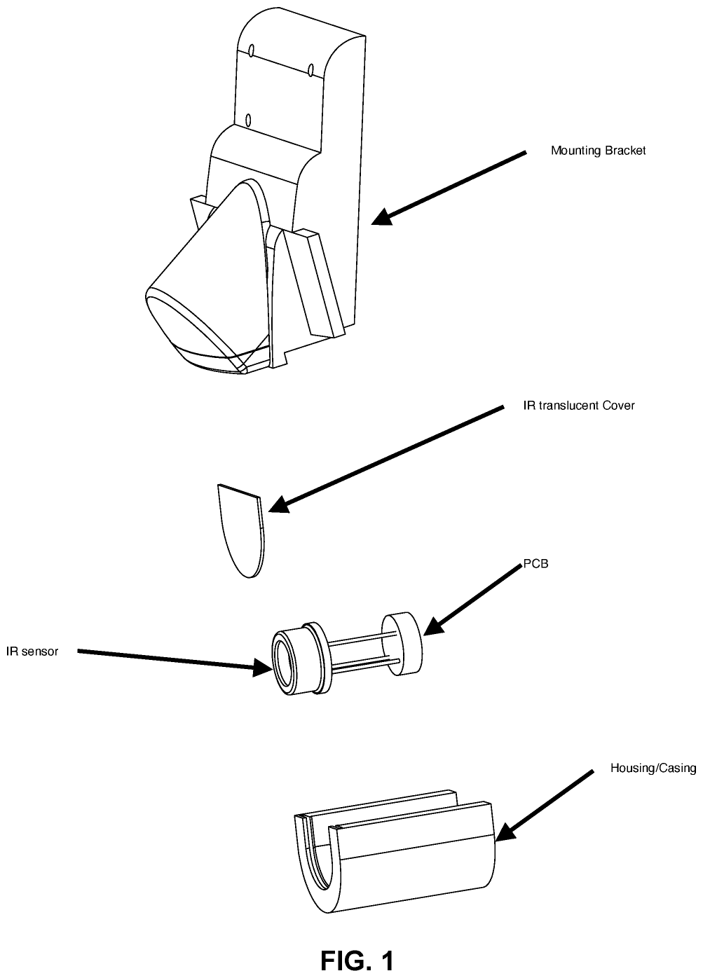

One embodiment of the temperature sensor is illustrated in FIG. 1, showing an IR sensor connected to a PCB, a mounting bracket, an IR translucent cover, and a housing.

In some embodiments, the temperature sensor is attached to the toilet seat or toilet rim in a fixed position directed toward body of a person sitting on the toilet seat.

In other embodiments, the temperature sensor further comprises components to rotate and/or telescope the temperature sensor to direct it to a part of the user or to the feces where temperature measurement is desired. In some of those embodiments, the components direct the temperature sensor to the anus and/or perineum of the user.

In other embodiments the temperature sensor consists of a one or two dimensional array of IR temperature sensors. The spacing and orientation of the array can be optimized without undue experimentation to address variability in seating location and the length of the perineum.

In order to direct the temperature sensor to a particular part of the user or to feces, various embodiments further comprise a lens operatively connected to a computing device. The lens and computing device can be dedicated to directing the movement of the temperature sensor and recording the temperature measurement. Alternatively, the lens and computing device can be part of an excreta analysis device that analyzes excreta during use of a toilet by the user, for example as described in PCT Patent Applications PCT/US2018/26618 and PCT/US2020/019383. As described therein, the excreta analysis device can be attached to, or integrated into, a toilet seat or toilet rim.

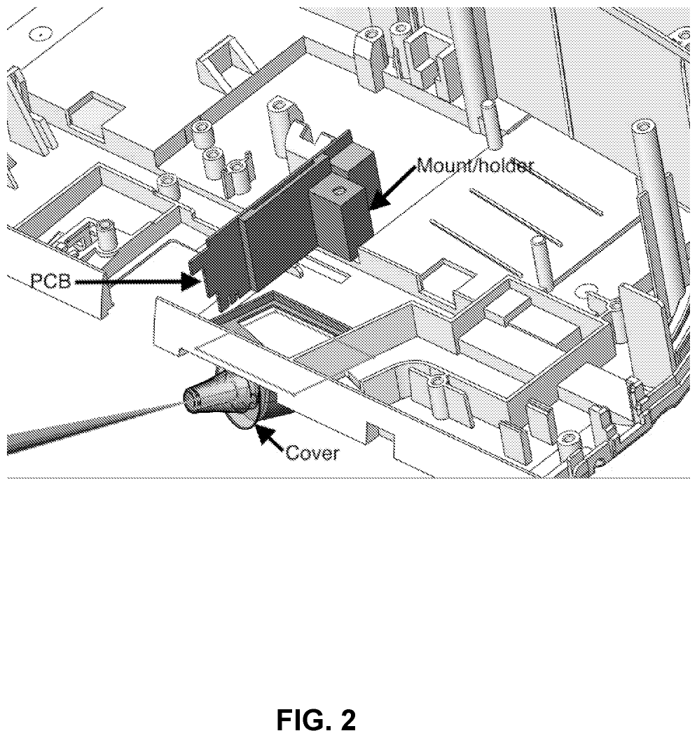

FIG. 2 shows an exemplary temperature sensor built into a structure that could be used to house components of an excreta analysis device, showing a mount/holder, a PCB, and an IR sensor, covered with a cover, e.g., a disposable cover.

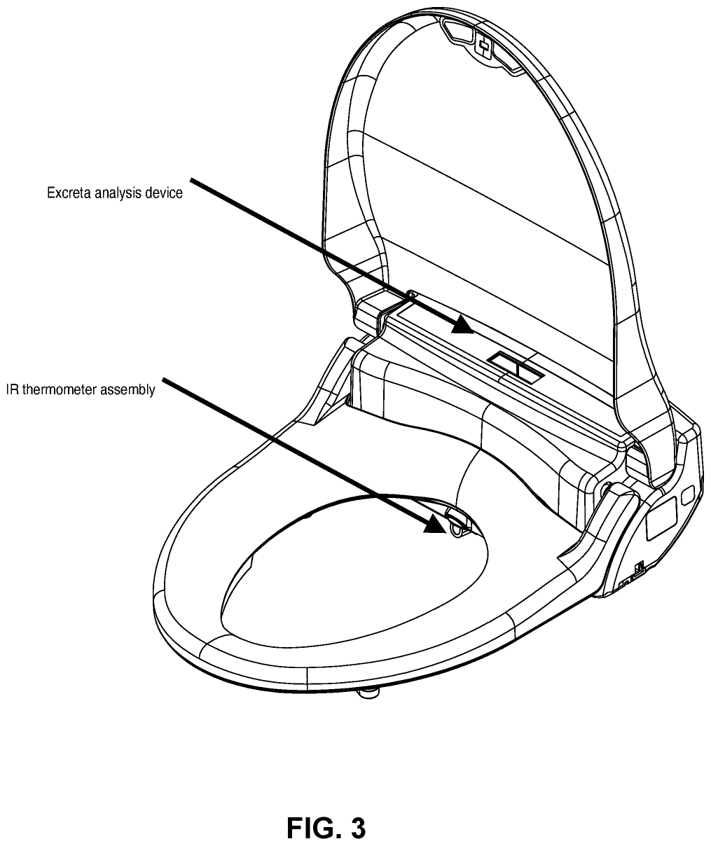

FIG. 3 shows an exemplary embodiment of such a device, where the IR thermometer assembly is integrated into a toilet seat with an excreta analysis device, as described in PCT/US2018/26618 and PCT/US2020/019383.

In some of those embodiments, the excreta analysis device comprises an image sensor, e.g., a time-of-flight camera, as described in PCT/US2018/26618 and PCT/US2020/019383. In such embodiments, the IR sensor and image sensor can be placed such that they do not interfere with each other if it is desired that both sensors can be used at the same time. Thus, one sensor can be centered on the toilet seat and the other sensor can be off-center, or each sensor can be off-center.

In some embodiments of the temperature sensor described above, a second temperature sensor is provided that is used to measure ambient temperature for calibration. The second temperature sensor is not narrowly limited to any particular sensor type. In some embodiments, the second temperature sensor is an IR temperature sensor. In other embodiments, the second temperature sensor is a thermistor, thermocouple, resistive temperature detector or other non-IR temperature sensor.

Also provided is a temperature sensor configured to fit on a bathroom appliance that measures a temperature of a user of the appliance without contacting the user. The temperature sensor and associated components can be any of those described above for the temperature sensor that fits under a toilet seat. For example, the temperature sensor of these embodiments can be measured using an any sensing method, including IR sensing or thermal imaging; additionally, the temperature sensor can further comprise a mounting bracket, an IR translucent cover, and/or a housing or casing.

In some embodiments, the bathroom appliance further comprises a bathroom use analysis device (BUAD) capable of transmitting data to a computing device, where the temperature sensor is also capable of transmitting data to the computing device.

As used herein, a bathroom use analysis device (hereinafter “BUAD”) is a device that measures a parameter of the use of a bathroom appliance such as a sink, a mirror, a tub, a bidet, a shower, a medicine cabinet, or a toilet, for example as described in PCT/US2020/019383.

In some embodiments, the bathroom appliance is a bidet, e.g., a hand-held bidet, where the user can direct the temperature sensor on the hand-held bidet to a particular part of the body.

In other embodiments, the bathroom appliance is a toilet, for example a toilet further comprising an excreta analysis device that measures and analyzes characteristics of excreta. In various embodiments, the excreta analysis device further comprises at least one sensor that generates data that can be used to detect and/or identify the user. In various embodiments, the toilet further comprises a bidet, for example an electronic bidet integrated into a toilet seat, wherein the temperature sensor is integrated into the washing nozzle.

Additionally provided is a temperature sensor configured to fit on a toilet seat that measures a temperature of skin contacting the seat. These embodiments are not narrowly limited to any specific type of temperature sensor. In various embodiments, the temperature sensor is an IR temperature sensor, a thermistor, a thermocouple, or a resistive temperature detector.

In some embodiments, the temperature sensor is integrated into the toilet seat. In various embodiments, the temperature sensor is capable of recording the temperature in a dataset and transmitting the dataset to a computing device. In some embodiments, the computing device can be dedicated to the temperature sensor. In other embodiments, the computing device is part of an excreta analysis device that analyzes excreta during use of a toilet by the user, e.g., as described in PCT/US2018/26618 and PCT/US2020/019383.

Further provided is a method of measuring temperature of a bathroom user, the method comprising using any of the above-described temperature sensors to measure the user's temperature.

### REFERENCES

Allegaert et al, 2014, Curr. Therapeutic Res. 76:34-38.

Blahd and Husney, 2019, Fever Temperatures, Healthwise, UW Health

Canadian Agency for Drugs and Technologies in Health, 2014, Rapid Response Report: Non-Contact Thermometers.

Mogensen et al., 2018, BMC Pediatrics 18:15.

Yao et al., 2007, Indoor Built Environ. 16:505-518.

PCT Patent Application PCT/US2018/026618.

PCT Patent Application PCT/US2020/019383.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

1. A sensor system, comprising:
a fitting mounted under a seat of a toilet;
a temperature sensor to measure temperature of an excrement expelled by the user without contacting the user or the excrement, wherein the temperature sensor further comprises a lens;
a circuitry in communication with the temperature sensor. the sensor configured to receive, store and transmit temperature data. received from the temperature sensor; and
a computing device to receive temperature data from the circuitry;
wherein the fitting attaches to a toilette fixture to receive the temperature sensor.

2-3. (canceled)

4. The system of claim 1, wherein the temperature sensor comprises one of a one dimensional IR sensors or two dimensional IR sensors.

5-7. (canceled)

8. The system of claim 1, wherein the toilette fixture further comprises a toilet seat or a toilet rim.

9-12. (canceled)

13. The system of claim 1, further comprising a disposable cover that fits over the temperature sensor.

14. The system of claim 1, further comprising an IR translucent cover.

15. (canceled)

16. The system claim 1, wherein the fitting further comprises a mounting bracket for mounting the sensor to the toilet seat or toilet rim.

17. The system of claim 1, wherein the fitting affixes the system to the toilet seat or toilet rim in a fixed position directed toward body of a person sitting on the toilet seat.

18. The system of claim 1, wherein the fixture further comprises components to rotate and/or telescope the temperature sensor to direct it to a part of the user or to the feces where temperature measurement is desired.

19. The system of claim 1, wherein the components direct the temperature sensor to the anus and/or perineum of the user.

20. (canceled)

21. The system of claim 1, wherein the lens and the computing device are a part of an excreta analysis device that analyzes the user excrement.

22-.23. (canceled)

24. The system of claim 1, further comprising a time-of-flight measurement component.

25. The system of claim 1, wherein the fixture further comprises components to rotate and/or telescope the temperature sensor to direct it to a part of the user or to the feces.

26. The system of claim 1, further comprising a second temperature sensor used to measure ambient temperature.

27-42. (canceled)

43. The system of claim 1, further comprising an excreta analysis device that measures and analyzes characteristics of excrement.

44. The system of claim 43, wherein the excreta analysis device further comprises at least one sensor that generates data to detect and/or identify the user.

45-52. (cancelled)
